SEQUENCE LISTING (13 sequences)

SEQ ID NO: 1 - protein, 13 aa, Artificial Sequence
  HIV-TAT protein
GRKKRRQRRRPPQ

SEQ ID NO: 2 - protein, 9 aa, Artificial Sequence
  TAT49-57 peptide
RKKRRQRRR

SEQ ID NO: 3 - protein, 12 aa, Artificial Sequence
  TAT49-57 peptide
YGRKKRRQRRRP

SEQ ID NO: 4 - protein, 13 aa, Artificial Sequence
  TAT49-57 peptide
GRKKRRQRRRPPQ

SEQ ID NO: 5 - protein, 16 aa, Artificial Sequence
  Penetratin
RQIKIWFQNRRMKWKK

SEQ ID NO: 6 - protein, 16 aa, Artificial Sequence
  Penetratin variant W48F
RQIKIFFQNRRMKWKK

SEQ ID NO: 7 - protein, 16 aa, Artificial Sequence
  Penetratin variant W56F
RQIKIWFQNRRMKFKK

SEQ ID NO: 8 - protein, 27 aa, Artificial Sequence
  Transportan
GWTLNSAGYLLGKINLKALAALAKKIL

SEQ ID NO: 9 - protein, 28 aa, Artificial Sequence
  Transportan-27
  MOD_RES LOCATION: (14)..(14): e-Cys
GWYLNSAGYLLGKXINLKALAALAKKIL

SEQ ID NO: 10 - protein, 23 aa, Artificial Sequence
  Transportan-22
  MOD_RES LOCATION: (14)..(14): e-Cys
GWYLNSAGYLLGKXINLKALAAL

SEQ ID NO: 11 - protein, 21 aa, Artificial Sequence
  Pep-1
KETWWETWWTEWSQPKKKRKV

SEQ ID NO: 12 - protein, 21 aa, Artificial Sequence
  Pep-2
KETWFETWFTEWSQPKKKRKV

SEQ ID NO: 13 - protein, 12 aa, Artificial Sequence
  HIV-TAT protein
GRKKRRQRRRPQ

(19) United States
(12) Patent Application Publication
(10) Pub. No.: US 2023/0173084 A1
(43) Pub. Date: Jun. 8, 2023

(54) PEPTIDE-BASED SYNTHETIC CHLORIDE ION TRANSPORTERS

(71) Applicant: Tavanta Therapeutics Hungary Incorporated, Budapest (HU)

(72) Inventors: István Mándity, Budapest (HU); József Maléth, Budapest (HU); Zoltán Varga, Budapest (HU); Nikolett Varró, Budapest (HU); Dorottya Bereczki-Szakál, Budapest (HU); Orsolya Basa-Dénes, Budapest (HU)

(73) Assignee: Tavanta Therapeutics Hungary Incorporated, Budapest (HU)

(21) Appl. No.: 17/782,603

(22) PCT Filed: Dec. 7, 2020
(86) PCT No.: PCT/IB2020/061590

§ 371 Date: Jun. 3, 2022

Related U.S. Application Data
(60) Provisional application No. 62/944,606, filed on Dec. 6, 2019.

(51) Int. Cl. A61K 47/64; A61K 47/55; A61P 11/00

(52) U.S. Cl. CPC A61K 47/645; A61K 47/55; A61P 11/00

(57) ABSTRACT

The present invention relates to the field of human therapy. In particular, the present invention relates to novel synthetic peptide-based chloride ion transporter and to compositions thereof, as well as methods of treating, reducing, inhibiting or controlling CFTR-mediated conditions in a subject, such as cystic fibrosis.

### CROSS-REFERENCE TO RELATED APPLICATIONS

This application is the U.S. National Stage of International Patent Application No. PCT/IB2020/061590, filed Dec. 7, 2020, which claims priority from U.S. Provisional Pat. Application No. 62/944,606, filed Dec. 6, 2019. The contents of these applications are incorporated herein by reference in their entirety.

### FIELD OF THE INVENTION

The present invention relates to the field of human therapy. In particular, the present invention relates to novel synthetic peptide-based chloride ion transporter and to compositions thereof, as well as methods of treating, reducing, inhibiting or controlling CFTR-mediated conditions in a subject, such as cystic fibrosis.

### SEQUENCE LISTING

The instant application contains a Sequence Listing which has been submitted electronically in ASCII format and is hereby incorporated by reference in its entirety. Said ASCII copy, created on Jan. 4, 2023, is name 127881_2100_ST25.txt and is 3,813 bytes in size.

### BACKGROUND OF THE INVENTION

Cell penetrating peptides (CPPs) or protein transduction domains (PTDs) are small peptides with less than 30 amino acid residues and of the appropriate size, charge and, polarity to pass through the cellular membrane. The main characteristics of these peptides include their ability to cross the cellular membrane using both endocytosis and energy-independent pathways, their high cellular permeability rates and their low cell toxicity and safety associated with little to no immunological response.

Currently, more than 1800 different CPPs have been reported and vast majority of them have been experimentally tested for different applications. CPPs are classified according to the type of cargo, their physicochemical properties (cationic, hydrophobic, amphipathic), their internalization mechanism and their structural features (linearity or cyclic nature).

Cell penetrating peptides (CPP) have been used for transporting various materials (peptides, proteins, DNA, RNA, etc.) through biological barriers such as plasma membranes (JP Richard, et.al., Journal of Biological Chemistry, 2003, 278, pp 585). Nevertheless, such systems have never previously been used or suggested for the transportation of ions through membranes.

Despite of the diversity of pathways and cell types targeted by CPP-based therapies, there are still no FDA approved CPP-conjugated drugs and several clinical trials have been discontinued to date. The problems associated with the use of CPP-conjugated drugs include: (1) in vivo stability issues due to frequent susceptibility to proteolytic degradation; (2) immunogenicity issues; (3) poor efficiency due to the drug’s failure to escape from endosomes after being internalized by cells; (4) toxicity due to the degradation of excipients; and (5) toxicity or poor efficiency due to the CPP’s lack of site specificity. (L Gomes dos Reis, D Traini, Expert Opinion on Drug Delivery, 2020, 17(5), pp 647; J Habault, JL Poyet, Recent Advances in Cell Penetrating Peptide-Based Anticancer Therapies, Molecules, 2019, 24 (5), pp 927).

Intensive research efforts have been devoted to developing synthetic ion channels using artificial compounds. The main strategies are related to the synthesis of unimolecular channels or the design of self-assembled supramolecular channels. These synthetic ion transporters or ion channels could complement the impaired or lost function of cellular ion channels (N Busschaert, PA Gale, Angewandte Chemie International Edition 2013, 52, pp 1374) and used for the treatment of channelopathies and related diseases.

Various artificial chloride transporters were developed with different molecular masses ranging from small organic molecules to supramolecular systems. These compounds either passively diffuse through the membrane with the chloride ion or form a channel in the membrane, facilitating passive ion transport. The general drawback of these compounds is their toxicity. It has been shown that some transporters increase intracellular sodium chloride concentrations, enhance cellular reactive oxygen species (ROS) levels, trigger the release of cytochrome c from the mitochondria and induce caspase activation, all of which results in apoptosis (SK Ko, et.al., Nat Chem 2014, 6, pp 885). On the other hand, it has been suggested that tumorous cells can be selectively killed by the utilization of artificial chloride transporters (D de Greñu, et.al.; Chemistry - A European Journal, 2011, 17, pp 14074).

These compounds can be used in oncology as perturbing the chemical gradients within cells, triggers apoptosis, leading to the death of tumorigenic cells. Chloride transporters are effective in the case of leukemia, lymphoma, myelofibrosis, and mastocytosis (S Parikh, et.al; Clinical Lymphoma Myeloma and Leukemia, 2010, 10, pp 285).

Respiratory diseases such as chronic obstructive pulmonary disease (COPD), asthma, cystic fibrosis (CF), bronchiectasis, tuberculosis, and lung cancer are leading causes of death, with numbers increasing yearly. There is an unmet need for new tools that could facilitate the development of new therapies for lung-related diseases as this has been recognised as the largest therapeutic failure. The field of bioactive molecules (peptides, proteins, and nucleic acids) is an area in which potential new treatments for respiratory diseases could be developed (L Gomes dos Reis, D Traini, Expert Opinion on Drug Delivery, 2020, 17(5), pp 647).

CF is the most common autosomal recessive genetic disease characterized by multiorgan pathology and significantly decreased life expectancy caused by the impaired function or expression of CFTR. In CF, chloride transport is impaired due to genetic mutations of the CFTR gene leading to absent, or diminished function of the cystic fibrosis transmembrane conductance regulator (CFTR) protein (BP O’Sullivan, SD Freedman, Lancet, 2009, 373, pp 1891). Recent therapeutic developments have significantly enhanced the life expectancy of patients with CF, yet the average age of death (usually caused by respiratory failure) is still 31.4 years (A Orenti, et al, ECFSPR Annual Report, 2016). In addition, 31% of the CF patients have chronic lung infections caused by Pseudomonas aeruginosa, whereas 83% of all CF patients need pancreatic enzyme replacement therapy, resulting in a significant burden to both the patients and healthcare systems.

There is thus a significant need for the development of synthetic chloride ion transporters that could be potentially utilized in channel replacement therapy to treat diseases associated with dysregulated anion transport - such as cystic fibrosis (CF).

Oral delivery of such therapeutic agents would be desirable, though this remains an unsolved challenge for drug formulators and drug delivery experts due to instability of the peptide-based bioactives in the GI tract, their low permeability and extremely rapid clearance (S Guptaet.al., Drug Delivery, 2013, 20, pp 237-246). Although cystic fibrosis is a systemic disease affecting - among others - the lungs, the digestive system and the reproductive system, lung infections and lung complications are the primary cause of death, accounting for up to 85% of the cases. (C Martin, et.al, Journal of Cystic Fibrosis, 2016, 15, pp 204-212). Abnormal mucus viscosity and production is known to contribute to CF pathogenesis (C Ehre et al, The International Journal of Biochemistry & Cell Biology, 2014, 52, pp 136-145). Moreover, hyper-concentrated mucus with increased airway adhesion is known to induce CF-like disease in animal models (M Mall et al, Nature Medicine, 2004, 10, pp 487-493). Analysis of the bronchoalveolar lavage fluid of young CF patients indicated that abnormally viscous mucus accumulation, with increased total mucin and inflammatory factor concentrations, drives the early pathogenesis of CF disease (CR Esther et al, Science Translational Medicine, 2019, 11, pp 1-11). The increase in mucus viscosity is due to the decreased cellular secretion of chloride ions, which results in an impaired fluid secretion and increased apical sodium absorption by the airway epithelial cells (H Li et.al., Current Opinion in Pharmacology, 2017, 34, pp 91-97). These alterations in ion transport ultimately result in the acidification and decreased height of the apical airway surface liquid.

In CF patients, cilial movement is impaired due to these alterations and the viscous mucus layer cannot be removed from the smaller airways. This results in chronic cough and increased probability and frequency of lung infections. Hydration therapy has been demonstrated to correct CF sputum samples to near-normal viscoelasticity, reinforcing the clinical findings that administration of hydrating agents yields beneficial results in patients with CF (BE Tildy and DF Rogers, Pharmacology, 2015, 95, pp 117-132). Therefore, a synthetic chloride ion transporter administered directly to the lungs could alleviate the symptoms associated with the highly viscous mucus layer (independent of the mutation causing the disease) by increasing the electrolyte levels of the layer and thus facilitating water transport out of the epithelial cells. Ultimately, this could lead to improved rheological properties of the mucus layer (D Schieppati, et.al, Respiratory Medicine, 2019, 153, pp 52-59). This is supported by the analysis of mucus samples: where it was shown that diluting (hydrating) mucus by a factor of 2 from 5.2% to 2.6% decreased the complex viscosity by a factor of eight (DB Hill et al, European Respiratory Journal, 2018, 52, pp 1-11).

Whilst VX (or ‘caftor’) compounds such as ivacaftor, lumacaftor, tezacaftor, elexacaftor and their combinations, as found in the marketed drug products Kalydeco, Orkambi, Symdeko and Trikafta, are effective in improving chloride ion transport, their use is limited to certain mutations of the CFTR gene. Moreover, there are numerous patients found to either be not responding to or not tolerating such caftor therapies. Treatment of these patients is an unmet medical need, in which synthetic chloride ion transporters may play a major role. Also, combining these universally effective ion channel transporters with established cystic fibrosis treatments, may result in improved therapeutic outcomes.

### SUMMARY OF THE INVENTION

1. A compound of Formula (X):

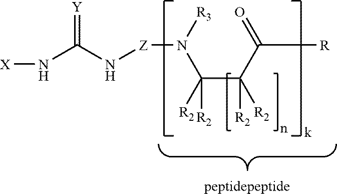

or pharmaceutically acceptable stereoisomers, enantiomers, diastereomers, racemic mixtures, polymorphs, tautomers, solvates, salts, esters, prodrugs or combinations thereof, wherein,
n= 0-10
k= 1-200
X= H, C1-10 alkyl or cycloalkyl, aryl, protecting group, C1-10 acyl, biotin, fluorescent and radioactive tracer, alkyl, cycloalkyl and acyl groups substituted with N, O, S, P, Se, Si, As, halides
Y= O, S, NH, CH₂, N-OR,
Z= C1-10 alkyl or cycloalkyl, aryl, protecting group, C1-10 acyl, biotin, fluorescent and radioactive tracer, alkyl, cycloalkyl and acyl groups substituted with N, O, S, P, Se, Si, As, halides
R= H, OH, O-alkyl, NH, N-alkyl, SH, S-alkyl, alkyl, alkenyl, alkynyl, NH-NH₂,
R2 = H, C1-10 alkyl or cycloalkyl, aryl, these substituted with N, O, S, P, Se, Si, As, halides, and form a ring system, and glycosylated, and
R3 = H, C1-10 alkyl or cycloalkyl, aryl, these ideally substituted with N, O, S, P, Se, Si, As, halides, and may form ideally a ring system, and may be glycosylated, and stereoisomers including enantiomers, diastereomers, racemic mixtures, mixtures of enantiomers or combinations thereof, as well as polymorphs, tautomers, solvates, salts, esters and prodrugs thereof.

2. The compound as recited in Point 1, wherein said peptide domain comprises one or more positively charged residues.

3. The compound as recited in Points 1 or 2, wherein said peptide domain comprises arginine or lysine side-chains.

4. The compound as recited in any of Points 1 to 3, wherein said peptide domain comprises one or more cell membrane penetrating domains (CPPs), such as cationic, amphipathic, hydrophobic or amphiphilic CPPs, selected from the group consisting of SP, pVEC, poly-arginine (arginine stretch), transportan, TAT, and penetratin, or variants thereof having at least having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to any of SEQ ID NOs: 1-13, and having cell penetrating activity, preferably selected from: residue 48-60 of TAT or penetratin, or variants thereof.

5. A compound as recited in any of Points 1 to 4, wherein said compound has Formula (I):

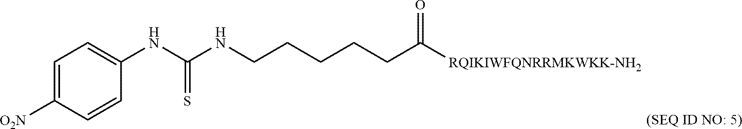

optionally wherein the molecular weight (MW) of the compound is 2537.4 Daltons.

6. A compound as recited in Point 5, wherein said compound is selected from pharmaceutically acceptable stereoisomers, enantiomers, diastereomers, racemic mixtures, polymorphs, tautomers, solvates, salts, esters, prodrugs or combinations thereof.

7. A compound as recited in any of Points 1 to 4, wherein said compound has Formula (II):

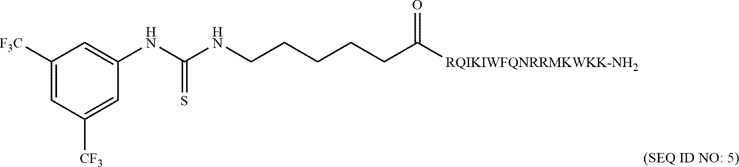

optionally wherein the molecular weight (MW) of the compound is 2628.4 Daltons.

8. A compound as recited in Point 7, wherein said compound is selected from pharmaceutically acceptable stereoisomers, enantiomers, diastereomers, racemic mixtures, polymorphs, tautomers, solvates, salts, esters, prodrugs or combinations thereof.

9. A compound as recited in any of Points 1 to 4, wherein said compound has Formula (III):

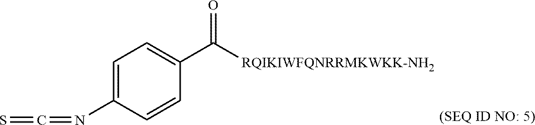

optionally wherein the molecular weight (MW) of the compound is 2405.3 Daltons.

10. A compound as recited in Point 9, wherein said compound is selected from pharmaceutically acceptable stereoisomers, enantiomers, diastereomers, racemic mixtures, polymorphs, tautomers, solvates, salts, esters, prodrugs or combinations thereof.

11. A compound as recited in Point 1, wherein said compound has Formula (IV):

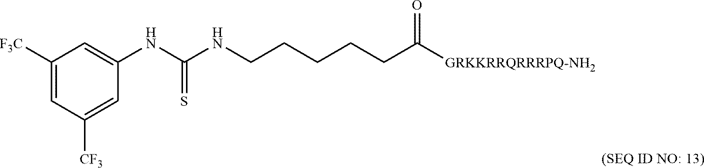

optionally wherein the molecular weight (MW) of the compound is 2004.4 Daltons.

12. A compound as recited in Point 11, wherein said compound is selected from pharmaceutically acceptable stereoisomers, enantiomers, diastereomers, racemic mixtures, polymorphs, tautomers, solvates, salts, esters, prodrugs or combinations thereof.

13. A compound as recited in any of Points 1 to 12, wherein said compound does not induce apoptosis or necrosis in a concentration range from 100 nM to 100 µM.

14. A compound as recited in any of Points 1 to 12, wherein said compound decreases the intracellular chloride ion concentration, when applied to HEK-293 cells at a concentration between 100 nM and 10 µM, optionally in a dose-dependent manner.

15. A compound as recited in any of Points 1 to 12, wherein said compound decreases the intracellular chloride ion concentration when applied to 3D pancreatic organoids at a concentration of 100 nM to 10 µM, optionally in a dose-dependent manner.

16. A compound as recited in any of Points 1 to 12, wherein said compound decreases the intracellular chloride ion⁻ concentration when applied to pancreatic ductal fragments in the absence of CFTR, at a concentration of 100 nM to 10 µM, optionally in a dose-dependent manner.

17. A pharmaceutical composition comprising a compound as recited in any of Points 1 to 12, and a pharmaceutically acceptable excipient or carrier.

18. A pharmaceutical composition comprising a compound as recited in any of Points 1 to 12, wherein said pharmaceutical composition is formulated for administration selected from the group consisting of oral, pulmonary, rectal, colonic, parenteral, intracisternal, intravaginal, intraperitoneal, ocular, otic, buccal, nasal, and topical administration; and/or formulated as a dosage form selected from the group consisting of liquid dispersions, gels, aerosols, ointments, creams, lyophilized formulations, tablets, capsules; and/or presented as a dosage form selected from the group consisting of controlled release formulations, fast melt formulations, delayed release formulations, extended release formulations, pulsatile release formulations, and mixed immediate release and controlled release formulations; and/or presented as an enema formulation, iontophoretic application, coating an implantable medical device; or combinations thereof.

19. A pharmaceutical composition according to any of Points 17 or 18, for use in the manufacture of a medicament.

20. A pharmaceutical composition according to any of Points 17 to 19, for use in the treatment, reduction, inhibition or control of viscous sputum or mucus associated with cystic fibrosis in a human subject, wherein said pharmaceutical composition increases the electrolyte content of said viscous mucus or sputum, such as chloride, optionally wherein said pharmaceutical composition is administered to the lungs of said human subject by pulmonary or aerosol delivery as a solution or suspension in a liquid vehicle, or as a dry powder.

21. A compound as recited in any of Points 1 to 12 or a composition as recited in any of Points 17 to 20 for use in therapy.

22. A compound as recited in any of Points 1 to 12, or a composition as recited in any of Points 17 to 20 for use in the treatment of CFTR-mediated diseases selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, mucopolysaccharidoses, chloride channelopathies such as myotonia congenita (Thomson and Becker forms), Bartter’s syndrome type III, Dent’s disease, hyperekplexia, epilepsy.

23. A method of treating, reducing, inhibiting or controlling viscous sputum or mucus associated with cystic fibrosis in a human subject, wherein said method comprises administration of a compound as recited in any of Points 1 to 12, or a composition as recited in any of Points 17 to 20 wherein said method increases the electrolyte content of said viscous mucus or sputum, such as chloride, optionally wherein said pharmaceutical composition is administered to the lungs of said human subject by pulmonary or aerosol delivery as a solution or suspension in a liquid vehicle, or as a dry powder.

24. A method of treating, reducing, inhibiting or controlling at least one sign or symptom of cystic fibrosis in a subject, wherein said method comprises administration of a therapeutically effective amount of one or more compounds as recited in any of Points 1 to 12 or a composition as recited in any of Points 17 to 20 to the human subject, optionally in combination with one or more therapeutic agents, wherein said sign or symptom is associated with the airways or respiratory system and includes one or more of the following: abnormally viscous mucus accumulation; increased total mucin content; elevated inflammatory factor concentration; decreased cellular secretion of chloride ions; impaired fluid secretion; increased apical sodium absorption by airway epithelial cells; acidification and decreased height of the apical airway surface liquid; chronic cough; chronic lung infection, and combinations thereof.

25. A pharmaceutical composition according to any of Points 17 to 20, or method according to any of Points 23 to 24 wherein said compound is preferably selected from Formula (I), (II), (III) and (IV).

### DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 shows the effect of Formula (II) and Formula (III) on cell viability in HEK 293 cells. Bar charts summarizing the effects of Formula (II) and Formula (III) on cell viability. Represented values highlight the percentage of total cell numbers. Results are visualized in % of total cell number (live/apoptotic/necrotic). As seen on the charts, no necrotic cell death was observed. For compounds of Formula (II) and Formula (III), a limited rate of apoptotic cell death was observed in 10 and 100 µM, respectively. However, the majority of cells survived the treatment. These results indicate that the tested compounds have no in vitro toxicity even in higher concentrations.

FIG. 2 shows the effect of Formula (I) - Formula (III) peptides on the intracellular Cl⁻ level in HEK 293 cells. Average traces of intracellular Cl^{—} levels of 4-6 experiments for each condition. HEK293 cells were perfused with HEPES-buffered extracellular solution. Administration of Formula (I) - Formula (III) induced a decrease in intracellular Cl^{—} levels (reflected by an increase in fluorescent intensity) due to the transport of Cl^{—} from the cytosol to the extracellular space. In these series of experiments Formula (I) - Formula (III) showed a dose-dependent effect and Formula (II) and Formula (III) had similar maximal effect in each concentration tested. Although Formula (I) also showed an in vitro effect on the intracellular Cl⁻, but due to the toxicity it was not investigated further.

FIG. 3 shows the effect of I-III peptides on the intracellular Cl^{—} level in HEK 293 cells. Bar charts of the maximal fluorescent intensity changes. II and III induced the highest maximal response, whereas all tested compounds showed dose-dependent effect.

FIG. 4 shows the effect of Formula (II) on the intracellular Cl^{—} level in pancreatic organoids. Average traces of intracellular Cl^{—} levels of 4-6 experiments for each condition. Pancreatic organoids were perfused with HEPES-buffered extracellular solution. Removal of extracellular Cl^{—} induced a decrease in intracellular Cl^{—} levels (reflected by an increase in fluorescent intensity) due to the activity of CFTR (Panel 1.). Administration of Formula (II) in 140 mM Cl- containing HEPES-buffered solution decreased the intracellular Cl^{—} level. Whereas in the absence of extracellular Cl^{—} the drop of intracellular Cl^{—} was remarkably higher.

FIG. 5 shows the effect of Formula (II) on the intracellular Cl^{—} levels in pancreatic organoids. Bar charts of the maximal fluorescent intensity changes. The effect of Formula (II) in the absence of extracellular Cl^{—} was comparable with the effect of CFTR.

FIG. 6 shows the effect of Formula (II) and Formula (III) on the intracellular Cl⁻ level in CFTR knockdown pancreatic ductal fragments. The ductal fragments were used to provide evidence that CLTR2 and CLTR-ITC can transport Cl^{—} in the presence or absence of CFTR protein. siGLO was used as a transfection control and siCFTR ductal fragments were treated with siRNA to knock down CFTR expression to model cystic fibrosis. CLTR2 and CLTR-ITC was able to transport Cl^{—} in siGLO (control) and siCFTR treated ductal fragments as well.

FIG. 7 shows the change in body weight of the animals during the treatment (A) and reduction in the lung parenchyma density in CFTR knockout mice and lung fibrosis (B-C).

### DETAILED DESCRIPTION OF THE INVENTION

Cell-penetrating peptides are small oligopeptides typically comprising between 5 and 30 amino acid residues. They are generally positively charged and are known to possess a random conformation in aqueous environment, however in the non-polar cell membrane, they show a tendency to fold into helical conformations (C Bechara, S Sagan, FEBS Letters, 2013, 587, pp 1693). They can pass through membranes either by a direct pathway or by a vesicular mode via endocytosis. Cell-penetrating peptides are known to transport various cargos ranging from small organic molecules to gene encoding DNAs (JP Richard, et.al, Journal of Biological Chemistry, 2003, 278, pp 585).

Synthetic ion transporters or ion channels could mimic the function of natural ion channels, thus rendering the unmet clinical need for channel replacement therapy feasible (N Busschaert, PA Gale, Angewandte Chemie International Edition, 2013, 52, pp 1374.). The development of lipid-bilayer chloride ion transporters for potential use in channel replacement therapy for the treatment of diseases caused by dysregulation of anion transport, such as cystic fibrosis (CF), is an area of current interest. In CF, the impaired chloride transportation is the main cause of the illness affected by the monogenic mutation of the cystic fibrosis transmembrane conductance regulator (CFTR) anion channel (BP O’Sullivan, SD Freedman, Lancet, 2009, 373, pp 1891).

CPP-based treatments may be combined with currently used therapies in CF, as the mechanism of action of each is completely different and result in synergy. CPP therapies can enhance the effect of mucolytic drugs and airway clearance techniques, as the application of CPP may increase the hydration of mucus. Synergistic effects are found in the combined application with VX compounds as CPP-based chloride ion transport is independent from the presence of functional CFTR in the membrane.

Various synthetic chloride transporters have been developed, with different molecular masses ranging from small organic molecules to supramolecular systems. These compounds either passively diffuse through the membrane with the chloride ion or form a channel in the membrane, opening the way for passive ion transport (N Busschaert, PA Gale, Angewandte Chemie International Edition 2013, 52, pp 1374).

Currently more than 2000 CFTR gene mutations have been described, whereas only 159 mutations have been characterized in terms of disease liability (R Bolia, et al., J Paediatr Child Health, 2018, 54, pp 609). The most common mutation type in 85% of patients worldwide is the deletion of phenylalanine at position 508 (F508del), however to date mutations are classified into seven different groups according to the CFTR defect caused (K De Boeck, MD Amaral, Lancet Respir Med, 2016, 4, pp 662). Class I mutations, which include frameshift, splicing or nonsense mutations that introduce premature termination codons; Class II mutations, which lead to misfolding and impaired protein biogenesis at the endoplasmic reticulum (ER); Class V mutations which result in reduced synthesis due to promoter or splicing abnormalities; and Class VI mutations that destabilize the CFTR channel in post-ER compartments and/or at the plasma membrane. Whereas Class III and IV mutations impair the gating and channel pore conductance respectively, thus selectively compromising CFTR function. In Class VII mutations, no mRNA can be detected. Current clinical treatment of CF is based on CFTR modulator therapy.

CFTR modulators include ivacaftor (Kalydeco®), lumacaftor/ivacaftor (Orkambi®), tezacaftor/ivacaftor (Symdeko®), and elexacaftor/tezacaftor/ivacaftor (Trikafta™). These drugs can increase the open state probability of CFTR and thus increase the ion efflux through the channel pore, or can promote the CFTR protein folding. Although these drugs have beneficial effects, their clinical use is restricted to limited patient populations with specific types of CFTR gene mutations. Channel replacement therapy with synthetic chloride ion transporters based on CPPs could overcome the severe limitation of current treatments, since such synthetic chloride transporters may promote chloride efflux across biological barriers even in the complete absence of CFTR protein. Therefore, chloride channel replacement therapy may provide mutation independent treatment, as the CFTR protein is not needed for Cl- ion transport, and could therefore be used early in patients, whereby their patient-specific mutations do not have to be characterized prior to commencement of such therapies. In addition, there are several patients with extremely rare mutations, which are not yet classified under the current mutation classes. CPP-based treatment may be applied in these patients without any clear restrictions.

Synthetic chloride ion transporter compounds of the present invention either passively diffuse through the membrane with the chloride ion or form a channel in the membrane, opening the way for passive ion transport.

Synthetic chloride ion transporters can be used in a mutation independent way, thus all CF patients may be treated using the compounds according to the invention.

Compounds described herein have the general formula as follows:

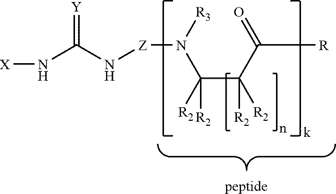

wherein,
n= 0-10;
k= 1-200;
X= H, C1-10 alkyl or cycloalkyl, aryl, protecting group, C1-10 acyl, biotin, fluorescent and radioactive tracer, alkyl, cycloalkyl and acyl groups can be substituted with N, O, S, P, Se, Si, As, halides;
Y= O, S, NH, CH₂, N-OR;
Z= C1-10 alkyl or cycloalkyl, aryl, protecting group, C1-10 acyl, biotin, fluorescent and radioactive tracer, alkyl, cycloalkyl and acyl groups can be substituted with N, O, S, P, Se, Si, As, halides;
R= H, OH, O-alkyl, NH, N-alkyl, SH, S-alkyl, alkyl, alkenyl, alkynyl, NH—NH₂;
R2 = H, C1-10 alkyl or cycloalkyl, aryl, these ideally substituted with N, O, S, P, Se, Si, As, halides, and may form ideally a ring system, and may be glycosylated, further including pharmaceutically acceptable stereoisomers, enantiomers, diastereomers, racemic mixtures, polymorphs, tautomers, solvates, salts, esters, prodrugs or combinations thereof..
R3= H, C1-10 alkyl or cycloalkyl, aryl, these ideally substituted with N, O, S, P, Se, Si, As, halides, and may form ideally a ring system, and may be glycosylated, further including pharmaceutically acceptable stereoisomers, enantiomers, diastereomers, racemic mixtures, polymorphs, tautomers, solvates, salts, esters, prodrugs or combinations thereof.

In an embodiment of the invention, the peptide domain(s) of the compounds described herein comprise one or more positively charged residues.

In another embodiment of the invention, said peptide domain(s) of the compounds described herein contain arginine or lysine side-chains.

In yet another embodiment of the invention, said peptide domain(s) of the compounds described herein are cell membrane penetrating peptides (CPPs), such as cationic, amphipathic, hydrophobic or amphiphilic CPPs.

In yet another embodiment of the invention, said peptide domain(s) of the compounds described herein are cell membrane penetrating peptide selected from one or more of the following:
a) HIV-TAT protein or a translocationally active derivative thereof, such as residue 48 to 60 of TAT: GRKKRRQRRRPPQ (SEQ ID NO: 1),
b) the TAT 49-57 peptide: RKKRRQRRR (SEQ ID NO: 2),
c) YGRKKRRQRRRP (SEQ ID NO: 3) (a longer peptide containing TAT49-57),
d) GRKKRRQRRRPPQ (SEQ ID NO: 4) (a longer peptide containing TAT49-57),
e) penetratin having the sequence RQIKIWFQNRRMKWKK (SEQ ID NO: 5),
f) penetratin variant W48F having the sequence RQIKIFFQNRRMKWKK (SEQ ID NO: 6),
g) penetratin variant W56F having the sequence RQIKIWFQNRRMKFKK (SEQ ID NO: 7),
h) penetratin variant having the sequence RQIKIWFQNRRMKFKK (SEQ ID NO: 7),
i) transportan having the sequence GWTLNSAGYLLGKINLKALAALAKKIL (SEQ ID NO: 8),
j) transportan-27 having the sequence GWYLNSAGYLLGK(e-Cys)INLKALAALAKKIL (SEQ ID NO: 9),
k) transportan-22 having the sequence GWYLNSAGYLLGK(e-Cys)INLKALAAL (SEQ ID NO: 10),
1) herpes simplex virus protein VP22 or a translocationally-active homologue thereof from a different herpes virus such as MDV protein UL49,
m) Pep-1, having the sequence KETWWETWWTEWSQPKKKRKV (SEQ ID NO: 11),
n) Pep-2, having the sequence KETWFETWFTEWSQPKKKRKV (SEQ ID NO: 12),
o) GRKKRRQRRRPQ (SEQ ID NO: 13).

In some embodiments, said peptide domain(s) of the compounds described herein may be TAT having the amino acid sequence of SEQ ID NO: 1, or a variant thereof, having at least 70%, 71%, 72%, 73%, 74%, 75%, 76%, 77%, 78%, 79%, 80%, 81 % 82%, 83%, 84%, 85%, 86%, 87%, 88%, 89%, 90%, 91%, 92%, 93%, 94%, or 95% identity to SEQ ID NO: 1 and having cell penetrating activity; or penetratin having the amino acid sequence of SEQ ID NO: 5, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to SEQ ID NO: 5 and having cell penetrating activity.

In some embodiments, said peptide domain(s) of the compounds described herein may comprise or consist of the amino acid sequence of any one of SEQ ID NOs: 2 to 4, or 6 to 13, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 2 to 4 or 6 to 13, and having cell penetrating activity.

In other embodiments of the invention, said peptide domain comprises one or more cell membrane penetrating domains selected from the group consisting of SP, pVEC, polyarginine (arginine stretch), transportan, TAT, and penetratin, or variants thereof having at least having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to any of SEQ ID NOs: 1 to 13, and having cell penetrating activity, preferably selected from: residue 48-60 of TAT or penetratin, or variants thereof.

In a further embodiment of the invention, the compounds described herein do not induce apoptosis or necrosis in a concentration range from 100 nM to 100 µM.

In another embodiment of the invention, the compounds of the present invention are amphipathic.

In an embodiment of the invention, the compounds of the present invention decrease the intracellular (Cl⁻) chloride ion concentration in a dose-dependent manner, optionally when applied into or onto an epithelial surface, optionally in a concentration range from 100 nM to 10 µM when applied to HEK-293 cells.

In another embodiment of the invention, the compounds of the present invention decrease the intracellular Cl^{—} concentration in a dose-dependent manner, optionally when applied to a tissue or organ, optionally in a concentration range from 100 nM to 10 µM when applied to 3D pancreatic organoids.

In an embodiment of the invention, the compounds of the present invention decrease the intracellular chloride ion concentration in a dose-dependent manner in a concentration range from 100 nM to 10 µM in pancreatic ductal fragments in the absence of CFTR.

In an embodiment of the invention, the compounds of the present invention decrease lung fibrosis and lung parenchyma density in cftr knockout mice in a dose of 1,64 mg/bwkg.

In an embodiment of the invention, the compounds of the present invention are useful for the treatment of CFTR-mediated diseases selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer’s disease, Parkinson’s disease, amyotrophic lateral sclerosis, progressive supranuclear plasy, Pick’s disease, several polyglutamine neurological disorders such as Huntington’s, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren’s disease, Osteoporosis, Osteopenia, bone healing and bone growth (including bone repair, bone regeneration, reducing bone resorption and increasing bone deposition), Gorham’s Syndrome; chloride channelopathies such as myotonia congenita (Thomson and Becker forms), Bartter’s syndrome type III, Dent’s disease, hyperekplexia, epilepsy; lysosomal storage disease, Angelman syndrome, and Primary Ciliary Dyskinesia (PCD), an inherited disorders of the structure and/or function of cilia, including PCD with situs inversus (also known as Kartagener syndrome), PCD without situs inversus and ciliary aplasia.

In another embodiment of the invention, the compounds of the present invention are useful in the treatment of cystic fibrosis patients presenting with one or more CFTR mutations, including Class I (e.g. G542X, W1282X, R553X, Glu831X), Class II (e.g. F508del, N1303K, I507del), Class III (e.g. G551D, S549N, V520F), Class IV (e.g. R117H, D1152H, R374P), or Class V mutations (e.g. 3849+10kbC>T, 2789+5G>A, A455E). The CF patient may present as a homozygotes or heterozygotes for any such CFTR mutation, e.g. F508del homozygote.

In a further embodiment of the invention, the compounds of the present invention are useful for the treatment of channelopathies, which are a heterogeneous group of disorders resulting from the dysfunction of ion channels located in the membranes of all cells and many cellular organelles, including diseases of the respiratory system (e.g., cystic fibrosis) and the urinary system (e.g., Bartter syndrome).

In a further embodiment of the invention, the compounds of the invention are prepared by elongating the peptide chain on a suitable gel resin such as TentaGel (R) RAM resin with a Rink amide linker. The coupling is preferably performed in two steps, namely, by dissolving Fmoc protected amino acid, the uronium coupling agent O-(7-azabenzotriazol-1-yl)-N,N,N′,N′-tetramethyluronium hexafluorophosphate (HATU) and N,N-diisopropylethylamine (DIPEA) in N,N-dimethylformamide (DMF) as solvent, under three hours of shaking in the first step and then the second coupling is performed with amino acid, HATU and DIPEA; then the resin is washed with DMF, methanol and DCM, and the washing is preferably followed by a deprotection step using 2% DBU and 2% piperidine in DMF in two steps with 15 and 5 minutes reaction times. After the coupling of the amino acids, the thiourea element is created, whereby the free N-terminus is reacted with specific isothiocyanates under alkaline conditions in DMF. After the completion of the sequence and thiourea construct the cleavage was carried out with TFA/water/dl-dithiothreitol (DTT)/TIS at 0° C. for 1 h.

In yet a further embodiment of the invention, there is provided a method of treating a channelopathy in a subject in need thereof, wherein said method comprises administration of a therapeutically effective amount of one or more compounds disclosed herein, to the subject, optionally in combination with one or more therapeutic agents.

In yet a further embodiment of the invention, there is provided a method of treating a CFTR-mediated disease selected from cystic fibrosis, asthma, COPD, smoke induced COPD, and chronic bronchitis fibrosis in a subject in need thereof, wherein said method comprises administration of a therapeutically effective amount of one or more compounds disclosed herein to the subject, optionally in combination with one or more therapeutic agents, preferably wherein said CFTR-mediated diseases is cystic fibrosis.

In a further embodiment of the invention, there is provided a method of treating cystic fibrosis in a human subject in need thereof, wherein said method comprises administration of a therapeutically effective amount of one or more compounds disclosed herein, to the human subject, optionally in combination with one or more therapeutic agents, wherein said subject is aged between 2 and 5 years of age, or between 6 and 11 years of age, or over 12 years of age.

In a further embodiment of the invention, there is provided a method of treating, reducing, inhibiting or controlling cystic fibrosis in a subject, wherein said method comprises simultaneously, separately or sequentially administering to the subject, (i) one or more therapeutic agents, and, (ii) a therapeutically effective amount of one or more compounds disclosed herein.

In a further embodiment of the invention, there is provided a method of treating, reducing, inhibiting or controlling at least one sign or symptom of cystic fibrosis in a subject, wherein said method comprises administration of a therapeutically effective amount of one or more compounds disclosed herein, to the human subject, optionally in combination with one or more therapeutic agents, wherein said sign or symptom is associated with the airways or respiratory system and includes one or more of the following: abnormally viscous mucus accumulation; increased total mucin content; elevated inflammatory factor concentration; decreased cellular secretion of chloride ions; impaired fluid secretion; increased apical sodium absorption by airway epithelial cells; acidification and decreased height of the apical airway surface liquid; chronic cough; chronic lung infection, and combinations thereof.

In a further embodiment of the invention, there is provided a method of treating, reducing, inhibiting or controlling at least one sign or symptom of cystic fibrosis in a subject, wherein said method comprises simultaneously, separately or sequentially administering to the subject, (i) one or more therapeutic agents, and, (ii) a therapeutically effective amount of one or more compounds disclosed herein, wherein said sign or symptom is associated with the airways or respiratory system and includes one or more of the following: abnormally viscous mucus accumulation; increased total mucin content; elevated inflammatory factor concentration; decreased cellular secretion of chloride ions; impaired fluid secretion; increased apical sodium absorption by airway epithelial cells; acidification and decreased height of the apical airway surface liquid; chronic cough; chronic lung infection, and combinations thereof.

In another embodiment of the invention is provided a pharmaceutical composition for use in the treatment, reduction, inhibition or control of viscous sputum or mucus associated with cystic fibrosis in a human subject, wherein said pharmaceutical composition increases the electrolyte content of said viscous mucus or sputum, such as chloride, optionally wherein said pharmaceutical composition is administered to the lungs of said human subject by pulmonary or aerosol delivery as a solution or suspension in a liquid vehicle, or as a dry powder.

In a further embodiment of the invention is provided a method of treating, reducing, inhibiting or controlling viscous sputum or mucus associated with cystic fibrosis in a human subject, wherein said method comprises administration of a compound, wherein said method increases the electrolyte content of said viscous mucus or sputum, such as chloride, optionally wherein said pharmaceutical composition is administered to the lungs of said human subject by pulmonary or aerosol delivery as a solution or suspension in a liquid vehicle, or as a dry powder.

### EXAMPLES

### Example 1

With Fmoc chemistry the peptide chain was elongated on TentaGel R RAM resin (0.19 mmol/g) (E Bayer, Angew. Chem. Int. Ed., 1991, 30, pp 113.) with a Rink amide linker on a 0.4 mmol scale manually. The coupling was performed in two steps. In the first step 3 equivalents of Fmoc protected amino acid, 3 equivalents of the uronium coupling agent O-(7-azabenzotriazol-1-yl)-N,N,N′,N′-tetramethyluronium hexafluorophosphate (HATU) (LA Carpino, Am. Chem. Soc., 1993, 115, pp 4379.) and 6 equivalents N,N-diisopropylethylamine (DIPEA) was used in N,N-dimethylformamide (DMF) as solvent with three hours of shaking. The second coupling was performed with 1 equivalent amino acid, 1 equivalent HATU and 2 equivalents of DIPEA. After the coupling steps, the resin was washed 3 times with DMF, once with methanol and 3 times with DCM. By these coupling conditions no truncated sequences was observed. The deprotection step was performed with 2% DBU and 2% piperidine in DMF in two steps with 15 and 5 minutes reaction times.

The resin was washed with the same solvents as described previously. After the coupling of the amino acids, the thiourea element was created. The free N-terminus was reacted with specific isothiocyanates under alkaline conditions in DMF. After the completion of the sequence and thiourea construct, the cleavage was carried out with TFA/water/dldithiothreitol (DTT)/TIS at 0° C. for 1 h. The cleavage has been performed with TFA/water/DL-dithiothreitol (DTT)/TIS (90/5/2.5/2.5) at 0° C. for 1 h. The purification was performed by reverse-phase HPLC, using a Phenomenex Luna C18 100 Ǻ 10 µm column (10 mm x 250 mm).¹¹⁷ The HPLC apparatus was made by JASCO and the solvent system used was as follows: 0.1% TFA in water; 0.1% TFA, 80% acetonitrile in water; linear gradient was used during 60 min, at a flow rate of 4.0 mL min⁻¹, with detection at 206 nm. The fractions purity was determined by analytical HPLC using a JASCO HPLC system with a Phenomenex Luna C18 100 Ǻ 5 µm column (4.6 mm x 250 mm) and the pure fractions were pooled and lyophilized. The purified peptides were characterized by mass spectrometry.

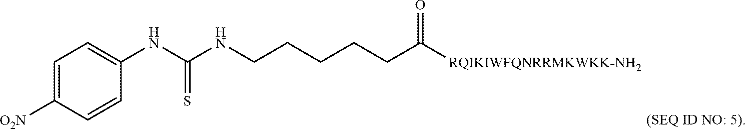

The molecular weight (MW) of the compound is 2537.4 Da; retention time is 12.8 min and its chromatographic properties: Gradient: 5->80% 25 min., A eluent: 0.1% TFA water, B eluent: 0.1% TFA 80% ACN 20% water (Column: Phenomenex Luna C18(2) 5 um, 100A, 250^{∗}4.6 mm).

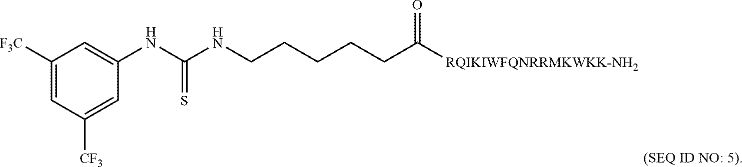

The molecular weight (MW) of the compound is 2628.4 Da; retention time is 14.9 min and its chromatographic properties: Gradient: 5->80% 25 min., A eluent: 0.1% TFA water, B eluent: 0.1% TFA 80% ACN 20% water (Column: Phenomenex Luna C18(2) 5um, 100A, 250^{∗}4.6 mm); typical IR wavenumbers for CF3 groups: 1132 cm⁻¹, 951.6 cm⁻¹, 887.2 cm⁻¹; HRMS: 2628.357 Da; 19F NMR (376.5 MHz, DMSO-d6, 4 mg/mL 298 K) -61.5 ppm

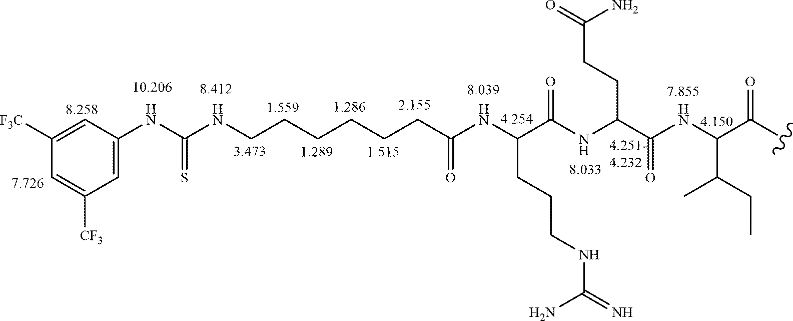

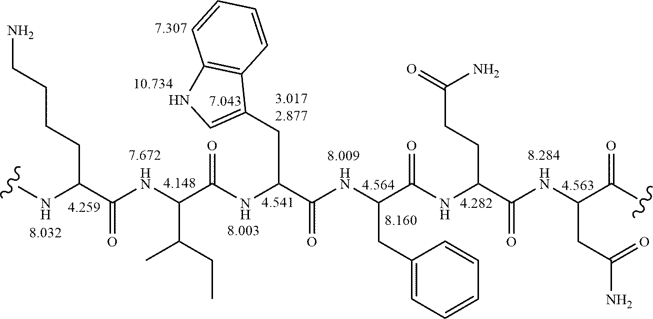

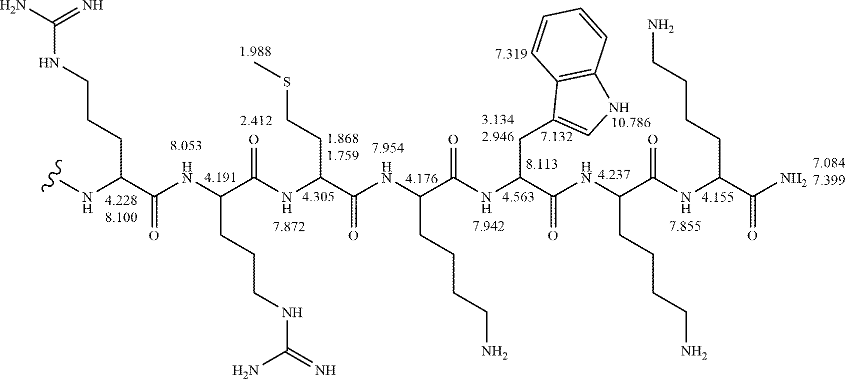

1H NMR signal assignment, DMSO-d6, 4 mg/mL 298 K.

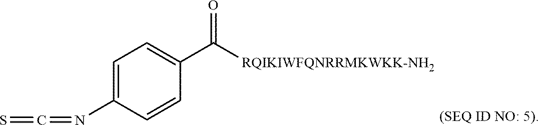

The molecular weight (MW) of the compound is 2405.3 Da; retention time is 13.5 min, and its chromatographic properties: Gradient: 5->80% 25 min., A eluent: 0.1% TFA water, B eluent: 0.1% TFA 80% ACN 20% water (Column: Phenomenex Luna C18(2) 5 um, 100A, 250^{∗}4.6 mm) typical IR wavenumbers for N=C=S groups: 1390 cm⁻¹, 1274 cm⁻¹, 1042 cm⁻1; for N=C bonds: 2095 cm⁻¹, HRMS: 2405.279 Da.

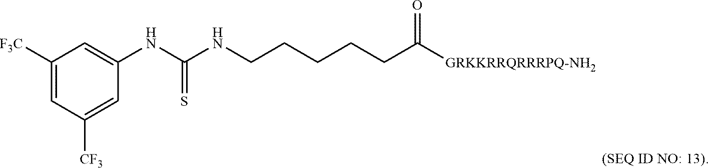

The molecular weight (MW) of the compound is 2004.4 Da; retention time is 13.9 min, and its chromatographic properties: Gradient: 5->80% 25 min., A eluent: 0.1% TFA water, B eluent: 0.1% TFA 80% ACN 20% water (Column: Phenomenex Luna C18(2) 5um, 100A, 250^{∗}4.6 mm).

### Example 2

### Effect of Compounds of Formula (I) - (Formula (III) on Cell Fate

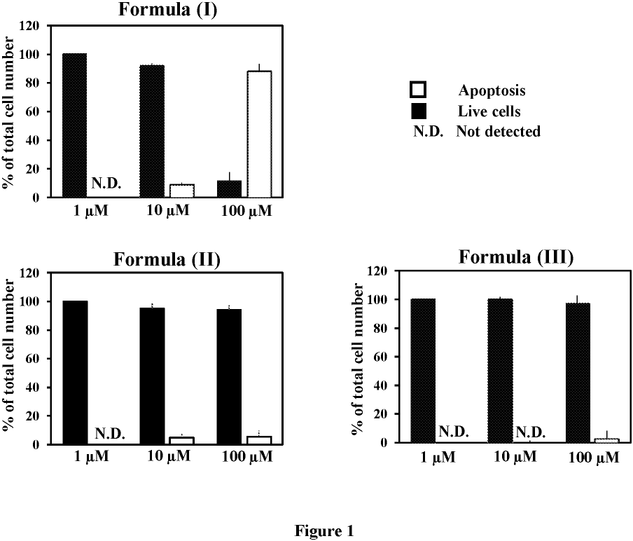

To investigate the effect of compounds according to the invention on cell fate, an apoptosis/necrosis detection kit was used according to the manufacturer’s instruction (Abcam Cat. No.: ab176750). Briefly, HEK-293 cells were incubated with various concentrations of CPPs for 30 min at 37° C. Cells were then washed 3 times and were incubated in 200 µL of Assay Buffer and loaded with CytoCalcein 450, Nuclear Green and Apopxin Deep Red at room temperature for 30-60 min. Following this, cells were washed and imaged. Images were captured using a Zeiss LSM880 confocal microscope with different channels and wavelengths according to each dye: CytoCalcein 450 (Ex/Em = 405/450 nm), Nuclear Green (Ex/Em = 490/520 nm) and Apopxin Deep Red (Ex/Em = 630/660 nm). For each condition, five images were captured and the total number of cells were counted by two independent investigators. Results are visualized in % of total cell number (live/apoptotic/necrotic) (FIG. 1). As can be seen, no necrotic cell death was observed. For compounds of Formula (I) and Formula (II), a limited rate of apoptotic cell death was observed in 10 and 100 µM, respectively. However, the majority of cells survived the treatment. These results indicate that the tested compounds have no in vitro toxicity even in higher concentrations.

Penetratin was used as control in concentrations from 1-100 µM, which had no effect on cell damage. In contrast, Formula (I) induced apoptosis in a concentration dependent manner, as demonstrated by the green plasma membrane signal, therefore this compound was not selected for further analysis. Formula (II) and Formula (III) on the other hand showed negligible toxicity even in 100 µM and 97.3% of the cells were viable after the incubation. This suggest that the applied compounds won’t damage the lung epithelial cells during the application, which can limit the side effects.

### Examples 3

### Effect of Formula (I) - Formula (III) on the Intracellular Cl⁻ Level in 2D HEK 293 Cells in Extracellular Cl⁻ Free Media

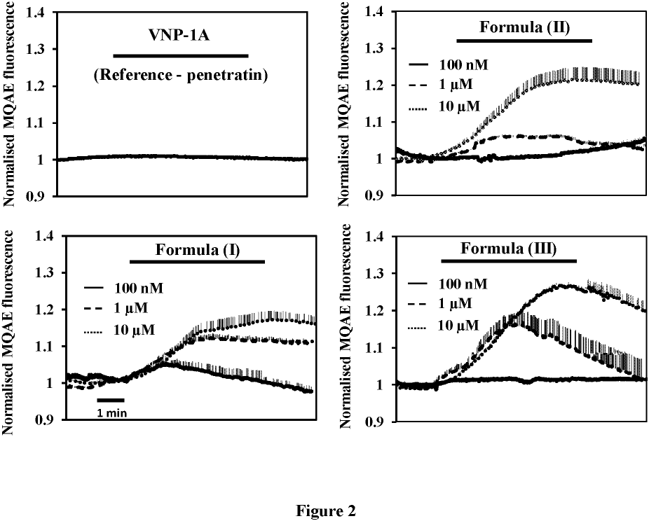

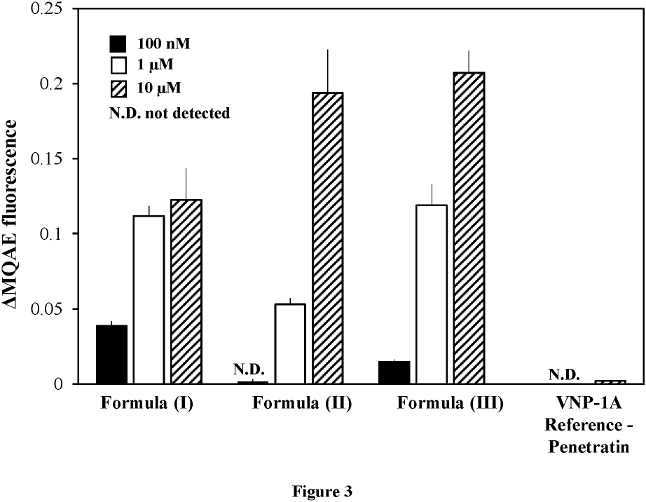

To assess the biological activity of CPPs changes of intracellular Cl^{—} level was measured by loading HEK-293 cells with 5 µM N-(Ethoxycarbonylmethyl)-6-Methoxyquinolinium Bromide (MQAE; ThermoFischer; Catalog number: E3101) for 30 min in the presence of 0.05% Pluronic F-127. Cells were bathed with Cl^{—} free external solution and treated with different concentrations of CPPs at 37° C. at the perfusion rate of 2-3 ml/min. Region of interests (ROIs) were determined by the xcellence softver (Olympus) and changes of Cl^{—} were determined by exciting the cells with an MT20 light source equipped with a 340/11 nm excitation filter. Excitation and emission wavelengths were separated by a 400 nm beam splitter and the emitted light was captured by a Hamamatsu ORCA- ER CCD camera. One measurement per second was obtained. During further analysis the fluorescence signals were normalized to the initial fluorescence intensity (F₁/F₀) and expressed as normalized MQAE fluorescence (FIG. 2). The maximal fluorescent intensity changes were calculated (FIG. 3.) Notably, the increase of normalised fluorescent intensity represents a decrease in the intracellular Cl^{—} concentration. N: 4-5 independent experiments/each tested condition.

All tested CPPs decreased the intracellular Cl^{—} concentration in a dose-dependent manner (FIGS. 2-3.). In 100 nM only Formula (I) showed a moderate response, whereas in 1 and 10 µM all synthetic chloride ion transporters decreased the intracellular Cl^{—} concentration. The highest response was achieved by Formula (II) and Formula (III). The control penetratin peptide had no effect.

### Examples 4

### Effect of Formula (II) on the Intracellular Cl⁻ Level in 3D Pancreatic Organoids in the Presence or Absence of Extracellular Cl^{—}

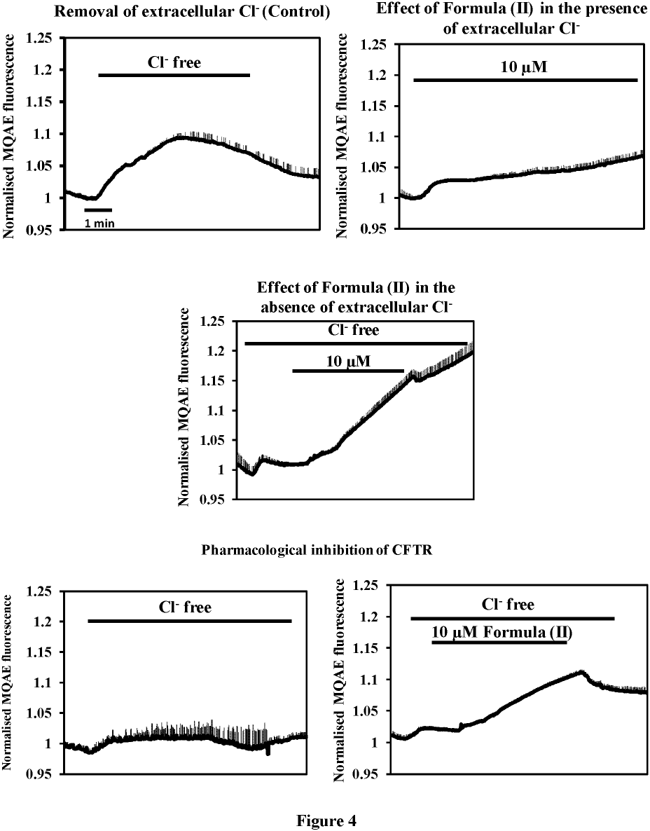

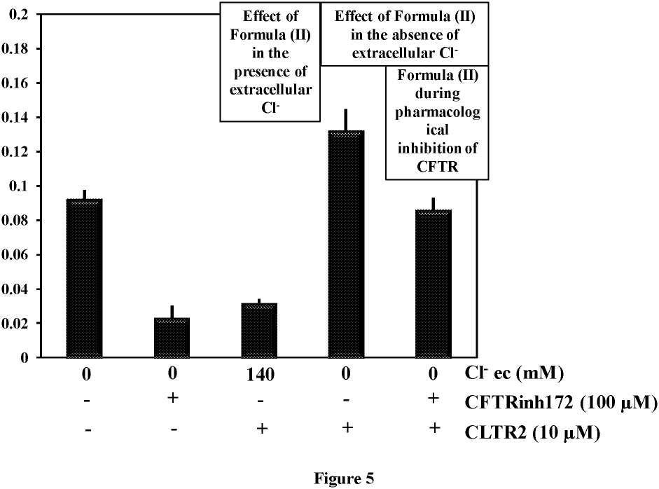

To test the Cl^{—} transporting capability of CPPs in primary 3D cells, organoids were loaded with MQAE as above described and bathed in standard HEPES buffered solution (FIG. 4.). Removal of extracellular Cl^{—} from the extracellular solution resulted in a decrease of intracellular Cl^{—}, most likely due to the Cl^{—} efflux from the cytosol via CFTR. As expected, pharmacological inhibition of CFTR with 100 µM CFTRinh172 almost completely abolished the fluorescent increase. Administration of Formula (II) induced intracellular Cl^{—} efflux in the presence and absence of extracellular Cl^{—}, which was not affected by the activity of CFTR (FIG. 5.). N: 4-5 independent experiments/each tested condition.

### Examples 5

### Effect of Formula (I) and Formula (III) on the Intracellular Cl⁻ Level in in CFTR Knockdown Pancreatic Ductal Fragments in the Absence of Extracellular Cl^{—}

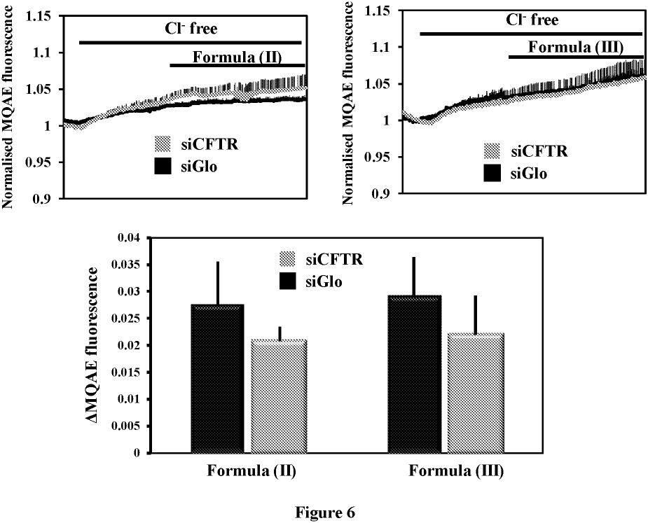

To test the biological activity of CPPs isolated mouse pancreatic ductal fragments were treated with siRNA to modify CFTR expression. For the transfection control indicator (SiGLO Green; Dharmacon; Catalog#: D-001630-01-50) and siCFTR (SMARTpool: ON-TARGETplus Cftr siRNA; Dharmacon; Catalog #: L-042164-00-0005) was used. Ductal fragments were kept in culture solution and transfected using Lipofectamine 2000 with siRNA duplexes after 12 h (20-40 nM/well) in 6-well plates in serum free medium according to the manufacturer’s protocol. The medium was changed to serum containing complete feeding medium 6 hours after adding the duplexes to the cells. The ductal fragments were harvested or used for measurements after 48 h (FIG. 6.).

Both Formula (I) and Formula (II) and induced Cl^{—} efflux in Cl^{—} free extracellular media in siGlo and siCFTR cells, indicating further that the tested synthetic chloride ion transporters transport Cl^{—} across the plasma membrane. N: 4-5 independent experiments/each tested condition.

### Example 6

### Effect of Formula (II) on the Severity of Lung Fibrosis in Cftr Knockout Mice

To assess the effect of in vivo administrated Formula (II) on lung histology parameters, this Example use Cftr^{tm1Unc}Tg(FABPCFTR)1Jaw/J mice (Jackson Laboratory, Stock No: 002364). FABP-hCFTR-CFTR bitransgenic mice harbor the FABP-hCFTR transgene [rat fatty acid binding protein 2, intestinal promoter directing expression of a human cystic fibrosis transmembrane conductance regulator (ATP-binding cassette sub-family C, member 7) gene] and a targeted knock-out mutation of the cystic fibrosis transmembrane conductance regulator homolog gene (Cftr). The mice used in this study were 8-12 weeks old and weighted 20-25 grams in the case of wild type (WT) animals and 15-17 grams in the case of CFTR knockout animals, the gender ratio was 1:1 for all groups. Experiments were carried out with adherence to the NIH guidelines and the EU directive 2010/63/EU for the protection of animals used for scientific purposes. The study was authorized by the National Scientific Ethical Committee on Animal Experimentation under license number XXI./1540/2020. Formula (II) was dissolved in physiological saline in a concentration of 10 µM. Treated mice received 400 µL Formula (II) dissolved in physiological saline solution in 5 minutes in a nebulizer in continuous oxygen flow (2L/min). Control animals received physiological saline as vehicle. The mice were grouped into 4 treatment groups as follows: wild type control (Group 1), CFTR knockout control (Group 2), wild type treated (Group 3), CFTR knockout treated (Group 4). Treatment was performed daily for 4 weeks. At the end of the experiments, mice received terminal anesthesia and the lungs were removed. Lungs were fixed for histology and trichrome staining was performed to assess lung parenchyma density and lung fibrosis. Sections were digitalized and fibrosis was scored as follows. 1388×1038 resolution pictures were taken with 10x and 40x magnification objectives with Zeiss ICc3 camera. Correction (of images taken with 40x objective) for background illumination was done in FIJI ImageJ package (v2.1.0/1.53e, Java 1.8..0_172 64 bit) as previously described (https://imagejdocu.list.lu/howto/working/how_to_correct_background_illumination_in_brigh tfield_microscopy). Analysis of anylin blue component of Massons trichrome fibrosis staining was done with Leica Aperio Image Scope (12.4.3.5008) with boult-in Positive Pixel Count v9 macro with hue threshold limit set to 0,607 with hue width of 0,12 (threshold limits 130-130,130-230). Positive pixels were counted as fibrosis, negative pixels were counted as tissue. Analysis of the air/tissue proportion was done with Leica Aperio Image Scope (12.4.3.5008) with built-in Positive Pixel Count v9 macro without hue limit (0-175;175-255). Areas of bronchi and blood vessels were excluded from the analyses.

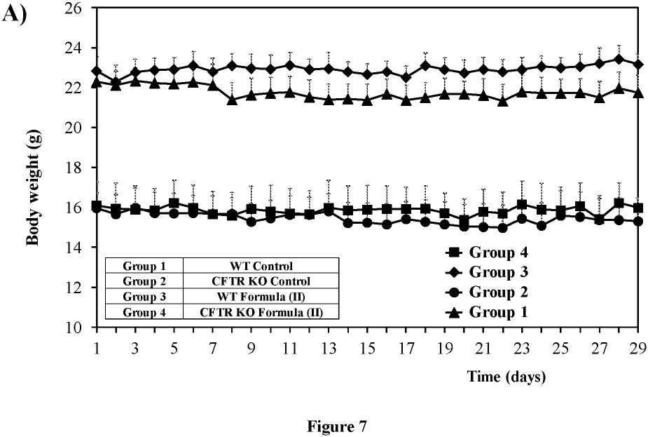

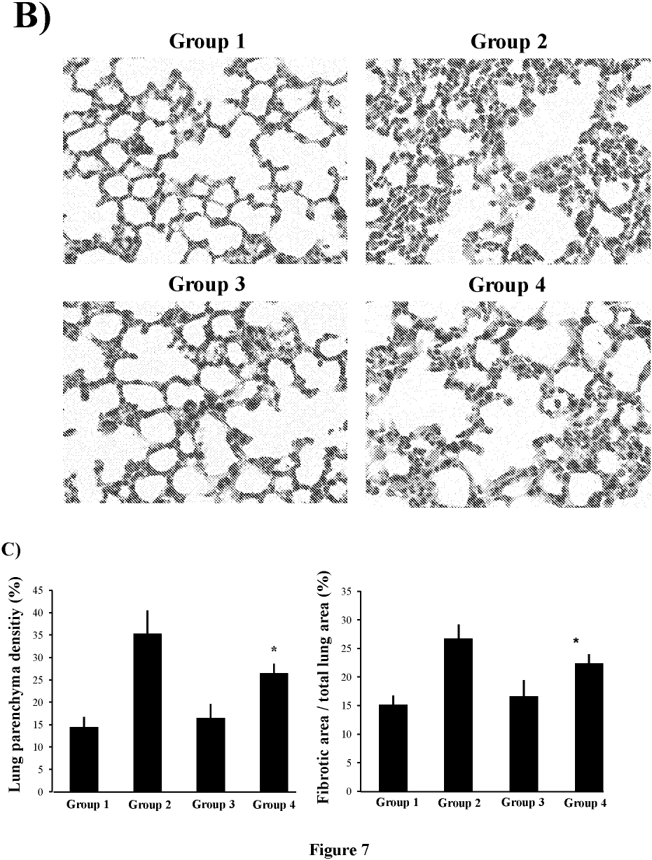

The body weight monitoring of the animals showed that the animals maintained their initial weights in all groups (FIG. 7.A). In vivo administration of Formula (II) significantly reduced the lung parenchyma density in CFTR knockout mice (35.2±5.2% vs 26.4±2.2%), and lung fibrosis (26.7±2.4% vs 22.4±1.6%) (FIG. 7. B-C) compared to the control group. During the treatment no adverse events were observed. No clinical signs of toxicity were observed for the treated animals.

1. A compound of Formula (X): 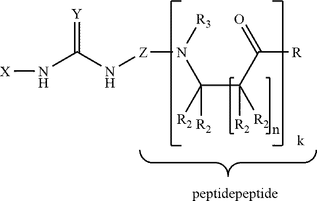
or pharmaceutically acceptable stereoisomers, enantiomers, diastereomers, racemic mixtures, polymorphs, tautomers, solvates, salts, esters, prodrugs or combinations thereof, wherein,
n= 0-10
k= 1-200
X= H, C1-10 alkyl or cycloalkyl, aryl, protecting group, C1-10 acyl, biotin, fluorescent and radioactive tracer, alkyl, cycloalkyl and acyl groups substituted with N, O, S, P, Se, Si, As, halides
Y= O, S, NH, CH₂, N-OR,
Z= C1-10 alkyl or cycloalkyl, aryl, protecting group, C1-10 acyl, biotin, fluorescent and radioactive tracer, alkyl, cycloalkyl and acyl groups substituted with N, O, S, P, Se, Si, As, halides
R= H, OH, O-alkyl, NH, N-alkyl, SH, S-alkyl, alkyl, alkenyl, alkynyl, NH-NH₂, R2 = H, C1-10 alkyl or cycloalkyl, aryl, these substituted with N, O, S, P, Se, Si, As, halides, and form a ring system, and glycosylated, and
R3 = H, C1-10 alkyl or cycloalkyl, aryl, these ideally substituted with N, O, S, P, Se, Si, As, halides, and may form ideally a ring system, and may be glycosylated, and stereoisomers including enantiomers, diastereomers, racemic mixtures, mixtures of enantiomers or combinations thereof, as well as polymorphs, tautomers, solvates, salts, esters and prodrugs thereof.

2. The compound as recited in claim 1, wherein said peptide domain comprises one or more positively charged residues.

3. The compound as recited in claim 1, wherein said peptide domain comprises arginine or lysine side-chains.

4. The compound as recited in claim 1, wherein said peptide domain comprises one or more cell membrane penetrating domains (CPPs), such as cationic, amphipathic, hydrophobic or amphiphilic CPPs, selected from the group consisting of SP, pVEC, poly-arginine (arginine stretch), transportan, TAT, and penetratin, or variants thereof having at least having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to any of SEQ ID NOs: 16, 17, 18, 19, 24 or 25, and having cell penetrating activity, preferably selected from: residue 48-60 of TAT or penetratin, or variants thereof.

5. A compound as recited in claim 1, wherein said compound has Formula (I): 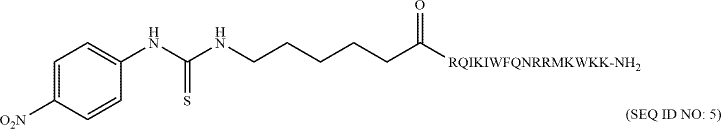
optionally wherein the molecular weight (MW) of the compound is 2537.4 Daltons.

6. A compound as recited in claim 5, wherein said compound is selected from pharmaceutically acceptable stereoisomers, enantiomers, diastereomers, racemic mixtures, polymorphs, tautomers, solvates, salts, esters, prodrugs or combinations thereof.

7. A compound as recited in claim 1, wherein said compound has Formula (II): 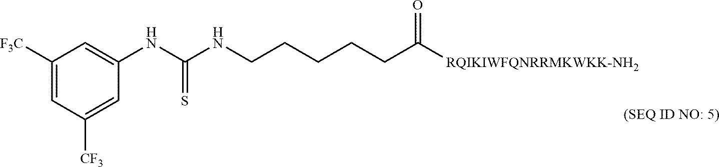
optionally wherein the molecular weight (MW) of the compound is 2628.4 Daltons.

8. A compound as recited in claim 7, wherein said compound is selected from pharmaceutically acceptable stereoisomers, enantiomers, diastereomers, racemic mixtures, polymorphs, tautomers, solvates, salts, esters, prodrugs or combinations thereof.

9. A compound as recited in claim 1, wherein said compound has Formula (III): 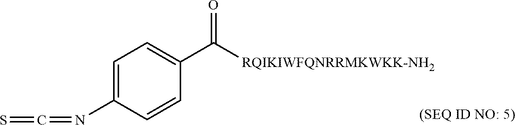
optionally wherein the molecular weight (MW) of the compound is 2405.3 Daltons.

10. A compound as recited in claim 9, wherein said compound is selected from pharmaceutically acceptable stereoisomers, enantiomers, diastereomers, racemic mixtures, polymorphs, tautomers, solvates, salts, esters, prodrugs or combinations thereof.

11. A compound as recited in claim 1, wherein said compound has Formula (IV): 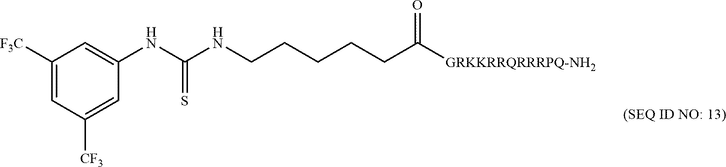
optionally wherein the molecular weight (MW) of the compound is 2004.4 Daltons.

12. A compound as recited in claim 11, wherein said compound is selected from pharmaceutically acceptable stereoisomers, enantiomers, diastereomers, racemic mixtures, polymorphs, tautomers, solvates, salts, esters, prodrugs or combinations thereof.

13. A compound as recited in claim 1, wherein said compound does not induce apoptosis or necrosis in a concentration range from 100 nM to 100 µM.

14. A compound as recited in claim 1, wherein said compound decreases the intracellular chloride ion concentration, when applied to HEK-293 cells at a concentration between 100 nM and 10 µM, optionally in a dose-dependent manner.

15. A compound as recited in claim 1, wherein said compound decreases the intracellular chloride ion concentration when applied to 3D pancreatic organoids or to pancreatic ductal fragments in the absence of CFTR at a concentration of 100 nM to 10 µM, optionally in a dose-dependent manner.

16. (canceled)

17. A pharmaceutical composition comprising a compound as recited in claim 1, and a pharmaceutically acceptable excipient or carrier, wherein said pharmaceutical composition is formulated for administration selected from the group consisting of oral, pulmonary, rectal, colonic, parenteral, intracisternal, intravaginal, intraperitoneal, ocular, otic, buccal, nasal, and topical administration; and/or formulated as a dosage form selected from the group consisting of liquid dispersions, gels, aerosols, ointments, creams, lyophilized formulations, tablets, capsules; and/or presented as a dosage form selected from the group consisting of controlled release formulations, fast melt formulations, delayed release formulations, extended release formulations, pulsatile release formulations, and mixed immediate release and controlled release formulations; and/or presented as an enema formulation, iontophoretic application, coating an implantable medical device; or combinations thereof.

18. (canceled)

19. (canceled)

20. A pharmaceutical composition according to claim 17, for use in the treatment, reduction, inhibition or control of viscous sputum or mucus associated with cystic fibrosis in a human subject, wherein said pharmaceutical composition increases the electrolyte content of said viscous mucus or sputum, such as chloride, optionally wherein said pharmaceutical composition is administered to the lungs of said human subject by pulmonary or aerosol delivery as a solution or suspension in a liquid vehicle, or as a dry powder.

21. (canceled)

22. (canceled)

23. A method of treating, reducing, inhibiting or controlling viscous sputum or mucus associated with cystic fibrosis in a human subject, wherein said method comprises administration of a compound as recited in claim 1 wherein said administration increases the electrolyte content of said viscous mucus or sputum, such as chloride, optionally wherein said pharmaceutical composition is administered to the lungs of said human subject by pulmonary or aerosol delivery as a solution or suspension in a liquid vehicle, or as a dry powder.

24. A method of treating, reducing, inhibiting or controlling at least one sign or symptom of cystic fibrosis in a subject, wherein said method comprises administration of a therapeutically effective amount of one or more compounds as recited in claim 1 to the human subject, optionally in combination with one or more therapeutic agents, wherein said sign or symptom is associated with the airways or respiratory system and includes one or more of the following: abnormally viscous mucus accumulation; increased total mucin content; elevated inflammatory factor concentration; decreased cellular secretion of chloride ions; impaired fluid secretion; increased apical sodium absorption by airway epithelial cells; acidification and decreased height of the apical airway surface liquid; chronic cough; chronic lung infection, and combinations thereof.

25. A pharmaceutical composition according to claim 17, wherein said compound is selected from Formula (I), (II), (III) and (IV).

26. A method according to claim 23, wherein said compound is selected from Formula (I), (II), (III), and (IV).
